(19) United States
(12) Patent Application Publication
(10) Pub. No.: US 2026/0091078 A1
(43) Pub. Date: Apr. 2, 2026

(54) OLIGOPEPTIDE THAT INHIBITS ANGIOGENESIS AND VASCULAR FUNCTION

(71) Applicants: Jakob TRIEBEL, Nuremberg (DE); Thomas Bertsch, Schwetzingen (DE); UNIVERSIDAD NACIONAL AUTONOMA DE MEXICO, Ciudad de Mexico (MX)

(72) Inventors: Jakob TRIEBEL, Nuremberg (DE); Thomas Bertsch, Schwetzingen (DE); Marid del Carmen Clapp Jiminez L., Juriqulla, Queretaro (MX); Juan Pablo Robles Alvarez, Santiago de Queretaro, Queretaro (MX); Maria Magdalena Zamora Corona, Juriquilla, Queretaro (MX); Gonzalo Martinez de la Escalera Lorenzo, Juriquilla, Queretaro (MX)

(21) Appl. No.: 17/778,396

(22) PCT Filed: Jul. 7, 2020
(86) PCT No.: PCT/EP2020/069154

§ 371 Date: May 19, 2022

(30) Foreign Application Priority Data
Nov. 20, 2019 (MX) MX/A/2019/013819

(51) Int. Cl. A61K 38/08; A61P 9/00; A61P 27/02; C07K 16/18

(52) U.S. Cl. CPC A61K 38/08; A61P 9/00; A61P 27/02; C07K 16/18

(57) ABSTRACT

An oligopeptide that inhibits angiogenesis and vascular function which oligonucleotide has a length of 3 to 7 amino acids and comprises or consists of the sequence X2-X3-X4, wherein X2 is a basic or amide amino acid, X3 is a small amino acid and X4 is a basic amino acid, charged at neutral pH or the sequence X1-X2-X3-X4, wherein X1 is a polar non-charged amino acid and X2, X3 and X4 are identical to X2, X3 and X4 in X2-X3-X4, or the sequence X1-X2-X3-X4-X5-X6-X7, wherein X1, X2, X3 and X4 are identical to X1, X2, X3 and X4 in X1-X2-X3-X4, X5 is a small amino acid, X6 is a hydrophobic amino acid and X7 is a hydrophobic amino acid.

### REFERENCE TO AN ELECTRONIC SEQUENCE LISTING

The contents of the electronic sequence listing (5309-37US_Sequence Listing.txt; Size: 4814 bytes; and Date of Creation: Jul. 8, 2025) is herein incorporated by reference in its entirety.

### TECHNICAL FIELD

The present invention refers to an antiangiogenic oligopeptide. The invention further refers to a pharmaceutical composition and a use of the oligopeptide.

### BACKGROUND

Angiogenesis is the formation of new blood vessels from the preexistent vasculature. It actively occurs during development determining the growth and differentiation of tissues. In the adult life, angiogenesis is restricted to reproductive events in females and to tissue repair due to wounds or fractures. Furthermore, the progression of high impact diseases such as cancer, diabetic retinopathy, and rheumatoid arthritis depends on the pathological stimulation of angiogenesis. Therefore, molecules with the ability to block angiogenesis have great therapeutic potential.

Several endogenous antiangiogenic factors have been characterized. Many of them are molecular fragments derived from the specific proteolysis of proteins with no activity on the angiogenesis process, including extracellular matrix and basal membrane proteins, as well as growth factors, cytokines, circulating proteins, and hormones.

Vasoinhibin is an antiangiogenic molecule generated when the hormone prolactin (PRL) loses its fourth alpha-helix after specific proteolytic cleavage by proteases including cathepsin D, matrix metalloproteases and bone morphogenetic protein 1. The remaining fragment conserving the N-terminal region and the first 3 helices of PRL was named vasoinhibin because of its inhibitory effects on angiogenesis and vascular functions, namely vasopermeability and vasodilation. Additionally, non-vascular actions of vasoinhibin have been reported, such as profibrinolytic effects, inflammatory actions, anxiolytic effects, and neural actions. Vasoinhibin is also known as prolactin of 16 kDa and is abbreviated PRL16K. Furthermore, vasoinhibin blocks different signaling pathways (Ras-Raf-MAPK, Ras-Tiam1-Rac1-Pak1, PI3K-Akt, and PLCγ-IP3-Enos) induced by pro-angiogenic factors (VEGF, Bfgf, bradykinin, and IL1β). Vasoinhibin blocks angiogenesis by inhibiting the proliferation, migration and survival of endothelial cells. Furthermore, vasoinhibin regulates vascular homeostasis by reducing vasodilation and vasopermeability by lowering the vascular production of nitric oxide. In animal studies, vasoinhibin induced depressive and anxiety related behavior.

It has been shown that vasoinhibin contributes to the physiological inhibition of angiogenesis in avascular organs and tissues, where angiogenesis is highly restricted, such as the retina and cartilage. Moreover, vasoinhibin actions play a role in the pathogenesis of angiogenesis-dependent diseases such as cancer, rheumatoid arthritis, diabetic retinopathy, and in peripartum cardiomyopathy and preeclampsia.

The molecular mechanism of vasoinh″bin action is only partially known. Vasoinhibin binds to endothelial cell membranes with high affinity and it was recently reported that vasoinhibin forms a multimeric complex in the endothelial cell surface with plasminogen-activator inhibitor (PAI-1), urokinase plasminogen-activator (Upa), and urokinase receptor (Upar). It has also been shown that vasoinhibin induces the apoptosis on endothelial cells through its specific binding to integrin alpha5 beta1.

Vasoinhibin is not a single molecular species but comprises a family of PRL fragments with different molecular masses determined by site of cleavage of the vasoinhibin-generating protease. These fragments include the first amino acid up to residues 123, 132, 139, 142, 147, 150, or 159 of the mature PRL. All of these isoforms inhibit angiogenesis, but their relative biological potencies are unknown Moreno-Carranza, B. et al., Sequence optimization and glycosylation of vasoinhibin: Pitfalls of recombinant production, Protein Expression and Purification. 161 (2019) 49-56 disclose the difficulties in expressing a peptide comprising the first 123 amino acids of human prolactin having good antiangiogenic properties in good yield.

From U.S. Pat. No. 7,300,920 B2 an anti-angiogenic peptide substantially identical to about 10 to about 150 consecutive amino acids selected from the N-terminal end of human placental lactogen, human growth hormone, or growth hormone variant Hgh-V is known, wherein the peptide (i) inhibits capillary endothelial cell proliferation and organization; (ii) inhibits angiogenesis in chick chorioallantoic membrane; and (iii) binds to at least one specific receptor which does not bind an intact full length growth hormone, placental lactogen, or growth hormone variant Hgh-V.

Nguyen, N.-Q.-N. et al., “Prolactin/growth hormone-derived antiangiogenic peptides highlight a potential role of tilted peptides in angiogenesis”, Proceedings of the National Academy of Sciences. 103 (2006) 14319-14324 show that tilted peptides exert antiangiogenic activity. Tilted (or oblique-oriented) peptides are short peptides known to destabilize membranes and lipid cores and characterized by an asymmetric distribution of hydrophobic residues along the axis when helical. It has been demonstrated that all these fragments possess a 14-aa sequence having the characteristics of a tilted peptide. The tilted peptides of human prolactin and human growth hormone induce endothelial cell apoptosis, inhibit endothelial cell proliferation, and inhibit capillary formation both in vitro and in vivo.

U.S. Pat. No. 7,655,626 B2 discloses a composition comprising an isolated antiangiogenic peptide or a fusion protein comprising a heterologous protein fused to the antiangiogenic peptide, wherein the peptide has antiangiogenic activity and consists of the amino acid sequence: X1-X2-X3-X4-X5-X6-X7-X8-X9-X10-X11-X12-X13-X14, wherein X1 is any amino acid residue compatible with forming a helix; X2 is an amino acid residue of: Leu; X3 is an amino acid residue of: Arg, Ser; X4 is an amino acid residue of: Ile, Leu; X5 is any amino acid residue compatible with forming a helix; X6 is an amino acid residue of: Leu, Val; X7 is an amino acid residue of: Leu, Ser; X8 is any amino acid residue compatible with forming a helix; X9 is any amino acid residue compatible with forming a helix; X10 is an amino acid residue of: Gln, Glu, Arg; X11 is an amino acid residue of: Ser; X12 is an amino acid residue of: Trp; X13 is an amino acid residue of: Leu, Asn; X14 is an amino acid residue of: Glu.

According to Robles, J. P. et al., Scientific Reports 8 (2018) 17111-17118, Vasoinhibin comprises a three-helix bundle and its antiangiogenic domain is located within the first 79 residues. Molecular dynamic simulation (MD) showed that the loss of the fourth α-helix (H4) exposes the hydrophobic nucleus of PRL and leads to the compression of the molecule into a three-helix bundle that buries the hydrophobic nucleus. It is further speculated that compression occurs by the movement of loop 1 (L1) and its interaction with α-helix 1 (H1) generating a new L1 conformation with electrostatic and hydrophobic surfaces distinct from those of PRL, that may correspond to a bioactive domain. Consistent with this model, a peptide sequence of 14 amino acids (residues 45 to 58) located in the early part of L1 of buffalo PRL was reported to exhibit antiangiogenic effects. This sequence was unveiled due to its 35.7% homology with human somatostatin, a known antiangiogenic factor. The authors found that a recombinant protein containing the first 79 amino acids comprising H1 and L1 of human PRL inhibited the proliferation and migration of endothelial cells and upregulated the vasoinhibin target genes, IL1A and ICAM1. This bioactivity was comparable to that of a conventional vasoinhibin having the 123 residues encompassing H1, L1, H2, L2, and H3 of human PRL. These findings indicated that the tilted peptide, absent in the 79 amino acid vasoinhibin, does not contain the most active biological determinant of vasoinhibin.

### SUMMARY

The problem to be solved by the present invention is to provide an alternative peptide which is able to exert the function of vasoinhibin by inhibiting angiogenesis and vascular function. It is a further object of the present invention to provide a recombinant protein, a recombinant nucleic acid, a pharmaceutical composition, the pharmaceutical composition for use in the treatment or prevention of a disease and a use of the peptide.

The problem of the present invention is solved by the features of claims 1, 3, 5, 11, 12, 14, 16 and 18. Embodiments of the invention are subject-matter of claims 2, 4, 6 to 10, 13, 15, 17 and 19 to 22.

According to a first alternative of the invention an oligopeptide that inhibits angiogenesis and vascular function is provided which oligopeptide has a length of 3 to 7 amino acids and comprises or consists of
the sequence X2-X3-X4, wherein
   X2 is a basic amino acid or an amide amino acid,
   X3 is a small amino acid and
   X4 is a basic amino acid, charged at neutral Ph
or
the sequence X1-X2-X3-X4, wherein
   X1 is a polar non-charged amino acid and
   X2, X3 and X4 are identical to X2, X3 and X4 in X2-X3-X4,
or
the sequence X1-X2-X3-X4-X5-X6-X7, wherein
   X1, X2, X3 and X4 are identical to X1, X2, X3 and X4 in X1-X2-X3-X4,
   X5 is a small amino acid,
   X6 is a hydrophobic amino acid and
   X7 is a hydrophobic amino acid.

In an embodiment of this first alternative of the invention
X1 is Thr, Ser, Asn, Glu, Gly, or Ala, in particular Thr,
X2 is His, Arg, Lys, Gln, or Asn, in particular His,
X3 is Ala or Gly, in particular Gly,
X4 is Arg or Lys, in particular Arg,
X5 is Gly, Ser, or Ala, in particular Gly,
X6 is Phe, Ala, Leu, Ile, Trp or Pro, in particular Phe and
X7 is Phe, Ala, Leu, Ile, Trp or Pro, in particular Ile.

According to a second alternative of the invention an oligopeptide that inhibits angiogenesis and vascular function is provided which oligopeptide has a length of 3 to 7 amino acids and comprises or consists of
the sequence X1-X2-X3, wherein
   X1 is an acid amino acid with negative charge at neutral Ph,
   X2 is a polar amino acid and
   X3 is an amino acid with positive charge at neutral Ph
or
the sequence X1-X2-X3-X4, wherein
   X1, X2 and X3 are identical to X1, X2 and X3 in X1-X2-X3 and
   X4 is a polar aromatic amino acid
or
the sequence X1-X2-X3-X4-X5-X6-X7, wherein
   X1, X2, X3 and X4 are identical to X1, X2, X3 and X4 in X1-X2-X3-X4,
   X5 is a polar amino acid,
   X6 is a hydrophobic amino acid and
   X7 is a hydrophobic amino acid.

In an embodiment of this second alternative of the invention
X1 is Asp or Glu, in particular Glu,
X2 is Gln, Asn, Ser or Thr, in particular Gln,
X3 is Arg or Lys, in particular Lys,
X4 is Tyr,
X5 is Gln, Asn, Ser or Thr, in particular Ser,
X6 is Phe, Ala, Leu, Ile, Trp or Pro, in particular Phe and
X7 is Phe, Ala, Leu, Ile, Trp or Pro, in particular Leu.

According to a third alternative of the invention an oligopeptide that inhibits angiogenesis and vascular function is provided which oligopeptide has a length of 7 amino acids and the sequence X1-X2-X3-X4-X5-X6-X7, wherein
X1 is the amino acid Thr, Asp or Glu,
X2 is the amino acid His or Gln,
X3 is the amino acid Gly or Lys,
X4 is the amino acid Arg if X3 is Gly or the amino acid Tyr if X3 is Lys,
X5 is the amino acid Gly or Ser,
X6 is the amino acid Phe and
X7 is the amino acid Ile or Leu.

In the context of the present disclosure, terms are to be understood as follows: The terms “amino acid” and “amino acid residue” may be used interchangeably, and are not to be understood limiting.

### Amino Acid:

Proteinogenic amino acid

### Amino Acid Residue:

Proteinogenic amino acid residue

### Amide Amino Acid:

Amino acids with amidated side chains such as asparagine (Asn) and glutamine (Gin).

### Polar Amino Acid:

Amino acid residue that forms hydrogen bonds as donor or acceptor. From the naturally occurring proteinogenic amino acid residues there are 10 polar amino acid residues: Two are negatively charged at neutral Ph, namely aspartic acid (Asp) and glutamic acid (Glu), three have a positive charge at neutral Ph, namely arginine (Arg), lysine (Lys), and histidine (His), and 5 are uncharged at neutral Ph, namely glutamine (GIn), asparagine (Asn), serine (Ser), threonine (Thr) and tyrosine (Tyr).

### Polar Aromatic Amino Acid:

Polar amino acid residue with an aromatic ring such as tyrosine (Tyr).

### Small Amino Acid:

Amino acid residues with less than 100 cubic Ångstrom (Å³) of volume such as alanine (Ala), glycine (Gly), and serine (Ser), but not cysteine (Cys) and not other amino acids having a bigger volume than cysteine.

### Hydrophobic Amino Acid:

Amino acid residue normally buried inside the protein core, such as phenylalanine (Phe), tryptophan (Trp), isoleucine (Ile), leucine (Leu), methionine (Met), valine (Val), alanine (Ala), and cysteine (Cys). These amino-acid residues are non-polar.

### Basic Amino Acid:

Amino acid residue with a positive charge at neutral Ph in the side chain that often forms salt bridges such as arginine (Arg), lysine (Lys), and histidine (His).

### Positively-Charged Amino Acid:

Basic amino acid.

### Acidic Amino Acid:

Amino acid residue with a negative charge at neutral Ph in the side chain that often forms salt bridges and include aspartic acid (Asp) and glutamic acid (Glu).

### Negatively-Charged Amino Acid:

Acidic amino acid.

### Conservative Replacement:

Replacement of an amino acid by another amino acid that belongs to the same of the above classes of amino acids, namely polar amino acid, polar aromatic amino acid, small amino acid, hydrophobic amino acid, basic amino acid, amide amino acid, positively-charged amino acid, acidic amino acid and negatively-charged amino acid. Conservative substitution groupings include, e.g., valine-leucine-isoleucine, lysine-arginine, alanine-valine, and asparagine-glutamine.

### X % Similarity to an Oligopeptide:

Percentage of amino acids of the total number of amino acids of the oligopeptide replaced by conservative replacement. For example, a 70% similarity to an oligopeptide having 10 amino acids means that 7 of the 10 amino acids of the oligopeptide are replaced by conservative replacement.

### Peptide

A compound consisting of 2 or more amino acid residues.

### Oligopeptide

A peptide consisting of less than 20 amino acid residues

### Polypeptide

A peptide consisting of at least 20 and less than 50 amino acid residues

### Protein

A peptide consisting of at least 50 amino acid residues

Finding the potent antiangiogenic activity and inhibitory activity of vascular function in such a small oligopeptide as that of the invention was surprising. The oligopeptide according to the invention is constituted by only 3 to 7 amino acids. The small size of the oligopeptide provided by this invention has the advantage that it makes the oligopeptide easy to produce, purify, handle, and formulate. In spite of its small size this oligopeptide exhibits the same, a better or at least a similar biological potency than vasoinhibin with respect to inhibition of angiogenesis and vascular function. The amino acid sequence is different to that of the ‘tilted’ peptide known from Nguyen, N.-Q.-N. et al. which peptide has a much lower biological potency that the oligopeptide according to the invention.

The ease of production Is an important advantage when considering the known difficulties in expressing a peptide comprising the first 123 amino acids of human prolactin in good yield such that the peptide has good antiangiogenic properties and the difficulties in the production of diverse other antiangiogenic proteins derived from prolactin. The small size of the oligopeptide of the present invention is an important advantage for its production that results in high yield and stability of the oligopeptide and low costs for its production.

The oligopeptide of the present invention is soluble in water or buffer such as Dulbecc″s phosphate-buffered saline, PH 7. It is soluble at a concentration of up to about 15 mg ML⁻¹. Solubility is a clear advantage over the known hydrophobic“tilte” peptide and over the whole vasoinhibin molecule which exposes hydrophobic patches on its surface which are able to reduce its solubility and to promote its precipitation.

Chemical modifications of the oligopeptide of the present invention can increase its half-life and resistance to the digestive tract. Such modifications include the incorporation of dextro amino acids or conversion to retro inverso peptides and cyclic peptides.

Because the oligopeptide of the present invention conserves the bioactive properties of vasoinhibin, it can be used to design and generate specific antibodies that discriminate between PRL and vasoinhibin allowing the sensitive and specific quantification of vasoinhibin for its use in clinical trials, diagnostics, and treatment.

Furthermore, the oligopeptide of the present invention has a direct inhibitory effect on proliferation and invasion of cancer cells. The oligopeptide of the present invention can inhibit both the proliferation and migration of endothelial cells and the proliferation and migration of cancer cells. This dual effect is an advantage over antiangiogenic drugs used for the treatment of cancer that just have vascular effects.

Furthermore, the oligopeptide of the present invention can be used in the treatment of angiogenic-dependent diseases, related or not related to reproduction. The smaller size, hydrophilic nature, and potency compared to that of vasoinhibin allows the production and formulation of effective drugs containing the oligopeptide and increases stability of the drugs.

The present invention includes the sequence of the oligopeptide according to the invention within a recombinant protein or another structure that can be used as carrier.

The oligopeptide of the present invention includes an oligopeptide, in particular an agonistic oligopeptide, which oligopeptide has a sequence of an oligopeptide as defined above or a sequence having at least 70%, in particular at least 80%, in particular at least 85%, in particular at least 90%, similarity to an oligopeptide as defined above and further has a modification at one terminal end or modifications at both terminal ends of its sequence or a substitution of one, more or all of its amino acids having D-conformation (a D-amino acid) by an amino acid or amino acids having L-conformation (an L-amino acid). The modification(s) may be acetylation of an N-terminal end and/or amidation of a C-terminal end of the oligopeptide or a covalent binding between an N-terminal amino acid and a C-terminal amino acid of the oligopeptide resulting in a cyclization of the oligopeptide.

The oligopeptide of the present invention may comprise or consist of a sequence of loop 1 or a sequence having at least 70%, in particular at least 80%, in particular at least 85%, in particular at least 90%, similarity to loop 1 which loop 1 is loop 1 of PRL, growth hormone, or placental lactogen. In particular, the oligopeptide of the present invention may consist of or comprise any of the following sequences or consist of or comprise a sequence having at least 70%, in particular at least 80%, in particular at least 85%, in particular at least 90%, similarity to any of the following sequences:

Sequence No. 1:
(SEQ ID NO 1)
Thr His Gly Arg Gly Phe Ile
Sequence No. 2:
(SEQ ID NO 2)
Glu Gln Lys Tyr Ser Phe Leu
Sequence No. 3:
(SEQ ID NO 3)
Asp Gln Lys Tyr Ser Phe Leu
Sequence No. 4:
(SEQ ID NO 4)
Thr His Gly Arg
Sequence No. 5:
(SEQ ID NO 5)
Glu Gln Lys Tyr
Sequence No. 6:
(SEQ ID NO 6)
Asp Gln Lys Tyr
Sequence No. 7:
His Gly Arg
Sequence No. 8:
Glu Gln Lys
Sequence No. 9:
Asp Gln Lys

The oligopeptide of the present invention may be fused to a carrier protein. The carrier protein can improve its efficiently, its localization, and/or its half-life.

The oligopeptide of the present invention, in particular the oligopeptide having 7 amino acids, may contain approximately 42.86% of neutral residues, 28.57% of basic or acid residues and no more than 28.57% of hydrophobic residues. Furthermore, this oligopeptide may have a hydrophobicity of more than +10 Kcal mol⁻¹, according to the experimental scale of Wimley, W. C., White, S. H., Experimentally determined hydrophobicity scale for proteins at membrane interfaces, Nature Structural Biology. 3 (1996) 842. In particular, hydrophobicity may remain in a range of +11.76 to +11.90 Kcal mol-1. Furthermore, the oligopeptide of the invention may have a particular distribution of hydrophobic residues grouped in the C-terminal end.

The oligopeptide of 7 amino acids of this invention has a basic amino acid which is charged at PH ˜7.4, such as Lys or Arg at positions X3 or X4. Furthermore, this oligopeptide may contain a basic amino acid positively charged at PH≤6, such as His, at position X2 and an acidic amino acid negatively charged at neutral PH at position X1, such as Asp or Glu.

The invention further concerns a recombinant protein comprising a sequence of an oligopeptide according to the invention.

The invention also concerns a nucleic acid, in particular a recombinant nucleic acid, which nucleic acid consists of or comprises a sequence coding for an oligopeptide according to the invention or a sequence complementary to this sequence. The recombinant nucleic acid may be contained in an expression vector.

The invention further concerns a pharmaceutical composition comprising at least one oligopeptide according to the invention and/or at least on recombinant protein according to the invention and/or at least one recombinant nucleic acid according to the invention.

According to an embodiment the pharmaceutical composition comprises a pharmaceutically acceptable carrier which is pharmaceutically acceptable for mammal administration, in particular humans. The pharmaceutically acceptable carrier can be a physiologic salt solution.

Any physiologically compatible formulation can be used for administration of the oligopeptide according to the invention. For example, the formulation may be an aerosol or a paste or it may comprise lipids. The concentration of the oligopeptide of the invention in the pharmaceutical composition may vary from around 0.1% w/w to 50% w/w.

The oligopeptide of the present invention can be administered in compositions having different dosage forms. For example, for oral administration, powders, tablets, pills, capsules or dragees, as well as liquid dosage forms such as suspensions or syrups may be used. For intraocular or parenteral administration, the liquid and sterile form may be used. Other inactive ingredients such as carriers or excipients, such as glucose, lactose, sucrose, mannitol, starch, cellulose and one or more of its derivatives, or a PH buffer, can be contained in the pharmaceutical composition of this invention, e.g. for stabilization of the pharmaceutical composition. The pharmaceutical composition may contain liposomes, including emulsions, micelles, or liquid crystals. The liposomes may be directed to a particular target using an antibody or molecule that recognizes this target.

The oligopeptide of the invention can be administered locally, regionally, topically, or systemically through injection, inhalation, suppository, transdermic, and ocular administrations, among others. A pharmaceutical composition containing the oligopeptide of this invention can be administered by an aerosol for nasal administration, for example. The oligopeptide of the invention can also be administered through a catheter that allow its delivery to an internal or a remote tissue. The pharmaceutical composition can also include encapsulated oligopeptide for protection and/or controlled and prolonged release of the oligopeptide. Such a pharmaceutical composition can be implanted near or at a specific target tissue. Appropriate formulas for the pharmaceutical composition have been reported in different references, such as Shayne Cox, Pharmaceutical Manufacturing Handbook, Wiley Online Books, Canada, 2008. doi:10.1002/9780470259818.

According to a further aspect of the invention the pharmaceutical composition according to the invention is for use in the treatment or prevention of an angiogenesis-dependent disease. Any oligopeptide according to the invention, any recombinant protein according to the invention and any recombinant nucleic acid according to the invention can be for use in the treatment or prevention of an angiogenesis-dependent disease. The angiogenesis-dependent disease may be cancer, a vasoproliferative retinopathy, a diabetic retinopathy, or rheumatoid arthritis.

The invention further concerns a use of an oligopeptide according to the invention or the recombinant protein according to the invention for the generation of antibodies. For this use the oligopeptide may comprise or consists of sequence His Gly Arg, Glu Gln Lys, or sequence Asp Gln Lys and the recombinant protein may comprise one of these sequences. The antibodies may be used in a diagnostic method performed in vitro. The diagnostic method may concern diagnosis of preeclampsia, peripartum cardiomyopathy, delayed fetus growth, a condition related to an abnormal blood pressure, a depressive disorder, an anxiety disorder, or an angiogenesis-dependent disease. An abnormal blood pressure is a blood pressure below or above normal blood pressure, i. e., hypotension or hypertension. An angiogenesis-dependent disease is a disease, where angiogenesis and/or vasopermeability and/or vasodilation are altered such as rheumatoid arthritis, vasoproliferative retinopathy, diabetic retinopathy, and cancer.

The present invention also concerns a pharmaceutical composition that contains 1, 2 or 3 oligopeptide(s) with the above-mentioned characteristics, separated or in combination. Furthermore, this invention concerns the recombinant production of a precursor of any of the oligopeptides mentioned above, as well as a fusion molecule that contain any of the sequences mentioned above.

The oligopeptide of the present invention can be used as immunizing agent for the generation of antibodies that recognize whole vasoinhibin but not PRL. These antibodies allow the quantification of endogenous levels of vasoinhibin in serum, in other biological fluids, and in tissues. The quantification of the endogenous levels of vasoinhibin is important because it has been shown that in diseased conditions, such as preeclampsia, peripartum cardiomyopathy, and diabetic retinopathy, vasoinhibin may contribute to their progression.

The present invention concerns different strategies for the generation of the oligopeptide according to the invention. For example, the oligopeptide could be generated recombinantly from a precursor or together with a fusion protein. Furthermore, peptide synthesis, which is the most viable strategy to generate the oligopeptide according to the invention, can be done using different known protocols.

In the following the invention is described by means of examples. In the examples, oligopeptides of the invention are presented for illustrative purpose only and are not to be understood as limiting the scope of the invention.

### BRIEF DESCRIPTION OF THE FIGURES

FIGS. 1A and 1B show schematically the location of the 7-amino acids oligopeptide THGRGFI according to the invention in the linear amino acid sequence of vasoinhibin (FIG. 1A) and the chemical structure of this oligopeptide at pH 7.4, wherein the oligopeptide is modified at its terminal ends (FIG. 1B).

FIGS. 2A and 2B show dose-response graphs comparing the biological potency of the 7 amino acids oligopeptide THGRGFI and the 123 amino acid vasoinhibin on the proliferation of endothelial cells stimulated with growth factors.

FIGS. 3A and 3B show the inhibitory effect of the 7 amino acid oligopeptide THGRGFI and that of vasoinhibin having 123 amino acids on the invasion of endothelial cells stimulated with VEGF.

FIG. 4 shows the change of expression of mRNA of vasoinhibin target genes, interleukin-1α (IL-1α) and intracellular adhesion molecule 1 (ICAM1) in endothelial cells in response to 100 nM of vasoinhibin having 123 amino acids or the oligopeptide THGRGFI.

FIGS. 5A and 5B show the inhibitory effect of 100 nM of the oligopeptide THGRGFI and of vasoinhibin having 123 amino acids on capillary formation of endothelial cells cultured on a Matrigel™ layer.

FIGS. 6A and 6B show the inhibition of vascular permeability by the oligopeptide THGRGFI and by vasoinhibin having 123 amino acids for a time of 120 minutes (FIG. 6A) and at 120 minutes (FIG. 6B).

FIG. 7 shows the inhibition of the vascular permeability of a monolayer of endothelial cells by the oligopeptide THGRGFI and by vasoinhibin having 123 amino acids for a time of 120 minutes in absence (control, Ctl) or presence of VEGF alone or in combination with the oligopeptide or vasoinhibin.

FIG. 8 shows in vivo inhibitory effect of VEGF-induced retinal vasopermeability by the oligopeptide THGRGFI and by vasoinhibin having 123 amino acids.

FIGS. 9A and 9B show the effects of the oligopeptide THGRGFI, of vasoinhibin having 123 amino acids and of 3 oligopeptides with scramble sequences of the amino acids contained in the oligopeptide THGRGFI on the proliferation of endothelial cells stimulated with growth factors, wherein amino acids that were not changed in their position are indicated in bold.

FIGS. 10A and 10B show the location the oligopeptide THGRGFI and of three oligopeptides of 7 amino acids having an overlapping sequence with this oligopeptide in the linear sequence of vasoinhibin (FIG. 10A) and the effects of these oligopeptides on the proliferation of endothelial cells in the presence of VEGF (FIG. 10B).

FIGS. 11A and 11B show the sequences of synthetic oligopeptides of 7 amino acids where each of the amino acids of the oligopeptide THGRGFI was substituted by alanine, which is indicated in bold (FIG. 11A) and the biological potency of these oligopeptides on the proliferation endothelial cells stimulated with growth factors.

FIGS. 12A and 12B show the sequence of oligopeptides of 7, 4 and 3 amino acids of the present invention (FIG. 12A) and their biological potency on the proliferation of endothelial cells stimulated with growth factors.

### DETAILED DESCRIPTION

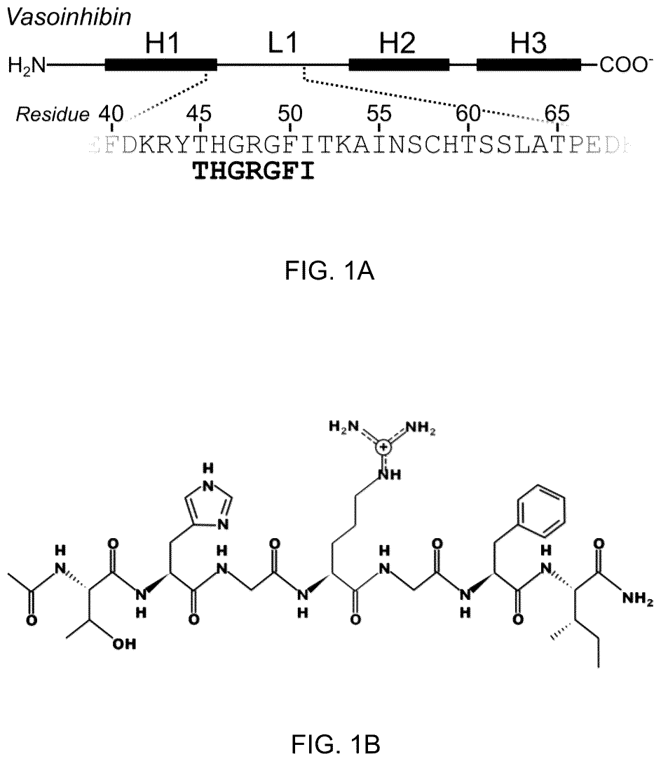

FIG. 1A shows the location of the 7 amino acids oligopeptide THGRGFI according to the invention in the linear amino acid sequence of vasoinhibin. The linear diagram of vasoinhibin extends from its N-terminal (H2N) to its C-terminal (COO⁻) end. The three main alpha-helixes (H1, H2 and H3) and the loop 1 (L1) connecting H1 and H2 are indicated. The sequence of residues 40 to 65 is zoomed to better appreciate the sequence. The location of the 7 amino acids oligopeptide THGRGFI is indicated in bold text and aligned with this sequence.

FIG. 1B shows an illustration of the primary structure of the oligopeptide THGRGFI according to the invention at pH 7.4. Amino and carboxyl terminal ends of this oligopeptide are acetylated and amidated, respectively.

### Example 1. Inhibition of Endothelial Cell Proliferation

The inhibitory effect of the oligopeptide of 7 amino acids, THGRGFI, that corresponds to SEQ ID NO 1, on the proliferation of immortalized bovine umbilical-vein endothelial cells (BUVEC E6E7) and of primary cultures of human umbilical-vein endothelial cells (HUVEC) was assessed and compared to the effect of a conventional 123 amino acid vasoinhibin.

On a 96-well plate treated for cell culture, BUVEC E6E7 and HUVEC cells were seeded at a density of ˜14,000 and ˜11,000 cells cm⁻², respectively. BUVEC E6E7 cells were maintained in F12K culture media with 10% (v/v) fetal bovine serum (FBS) and the HUVEC in F12K media supplemented with 20% of FBS, 100 μg ml⁻¹ heparin and 25 μg ml⁻¹ of endothelial cell growth supplement (ECGS). After 24 hours, the cells were starved with FBS-reduced media (0.1% of FBS for BUVEC E6E7 and 0.5% for HUVEC) for 16 hours to synchronize cells in phase GO of the reproductive cycle. After this time, FBS and heparin were replenished only to HUVEC. The cells were then treated with vasoinhibin or the oligopeptide THGRGFI in concentrations ranging from 0.001 to 100 nM for 24 hours in the presence of 10 μM of the thymidine analog 5-Ethynyl-2′-deoxyuridine (EdU) and 50 ng ml⁻¹ VEGF for the BUVEC E6E7 or the combination of 25 ng ml⁻¹ VEGF and 20 ng ml⁻¹ bFGF for HUVEC. At the end of the experiment, cells were fixed with 4% paraformaldehyde, permeabilized with 0.5% Triton X-100 in TBS1x, and stained to detect the newly synthetized DNA by the incorporation of EdU using the “click” assay (which involves a copper catalyzed reaction to covalently bind fluorescent azide to the EDU incorporated to DNA). Total DNA was counterstained with Hoechst 33342 and the “click”-stained nuclei were quantified and plotted relative to the total number of Hoechst 33342-stained nuclei.

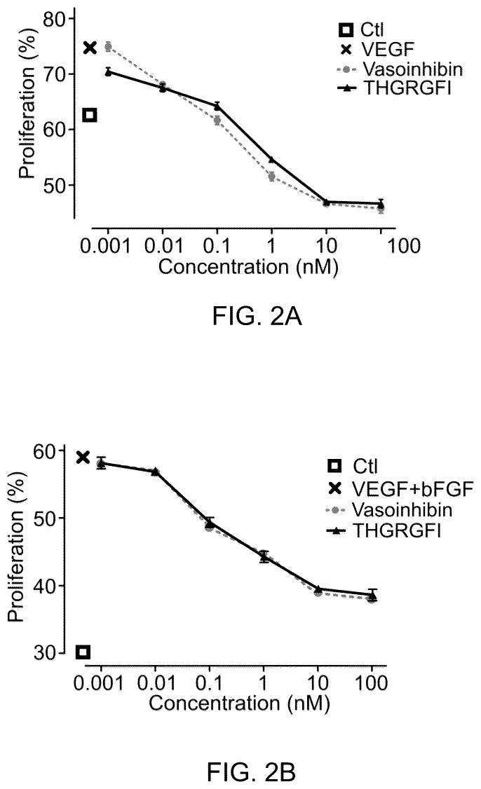

Results are illustrated in FIGS. 2A and 2B which show dose-response graphs comparing the biological potency of oligopeptide THGRGFI and the 123 amino acid vasoinhibin with respect to the proliferation of immortalized bovine umbilical-vein endothelial cells (BUVEC E6E7) stimulated with 50 ng mL⁻¹ VEGF (FIG. 2A) and with respect to the primary cultures of the human umbilical-vein (HUVEC) stimulated with a combination of VEGF (25 ng mL⁻¹) and bFGF (20 ng mL⁻¹) (FIG. 2B).

The proliferation of endothelial cells (BUVEC E6E7 and HUVEC) was inhibited in a dose-response manner by vasoinhibin and the oligopeptide THGRGFI. Both inhibitory agents had activity on both types of endothelial cells at the same effective dose of ˜1 nM (EC₅₀≈1 nM) (FIGS. 2A and 2B), similar to the previously reported potency of vasoinhibin. This result confirms that the 7-amino acid oligopeptide THGRGFI conserves the vasoinhibin potency with respect to inhibition of proliferation of endothelial cells. The oligopeptide exhibits a similar and robust dose-response behavior as vasoinhibin.

### Example 2. Inhibition of the Invasive Migration of Endothelial Cells

Vasoinhibin is able to inhibit the migration and invasion of endothelial cells by mechanisms that include the inactivation of Ras-Tiam1-Rac1-Pak1 pathway, the inactivation of the urokinase type plasminogen activator (uPA) due to the increased expression of plasminogen activator inhibitor-1 (PAI-1), and the inactivation of endothelial nitric oxide synthase (eNOS). For testing if the oligopeptide of the invention THGRGFI conserves the inhibitory properties on the invasive migration of endothelial cells, a migration assay was performed using a permeable ‘transwell’ support with a Matrigel™ matrix and conditioned media as chemoattractant.

Endothelial cells were seeded over 100 μl Matrigel™ matrix (380 ng μl⁻¹) on a permeable ‘transwell’ support in a transwell chamber having an area of 0.33 cm² and 8 μm of pore-diameter at a density of 30,000 and 14,000 cells cm⁻² for BUVEC E6E7 and HUVEC cells, respectively. In the upper (luminal) compartment the cells were maintained with starving media F12K having 0.1 or 0.5% FBS for BUVEC E6E7 and HUVEC, respectively. In addition, HUVEC cells were maintained with 100 μg ml⁻¹ heparin. In the inferior abluminal compartment, filtered (0.22 μm) conditioned media from 3T3-L1 cells (obtained by culturing 3T3-L1 cells in DMEM—10% FBS for 48 hours) and 50 ng ml⁻¹ of VEGF were used as chemoattractant. After 24 hours the media from both compartments and the luminal cells were removed. The abluminal cells were fixed with 100% MeOH for 10 minutes, permeabilized with TBS1x-0.5% Triton X-100, and stained with Hoechst 33342. The total number of cells in the abluminal compartment indicates the invasive activity of endothelial cells.

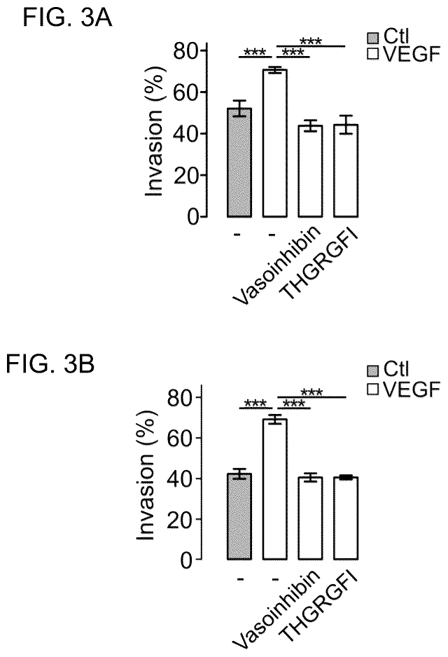

Results are shown in FIGS. 3A and 3B. The inhibitory effect of 100 nM of the oligopeptide THGRGFI on the invasion of immortalized endothelial cells from the bovine umbilical vein (BUVEC E6E7) (FIG. 3A) or primary cells from the human umbilical vein (HUVEC) (FIG. 3B) stimulated in each case with VEGF (50 ng mL⁻¹) was compared with the inhibitory effect of 100 nM of the 123 amino acid vasoinhibin (***P<0.001).

Both, the vasoinhibin and the oligopeptide THGRGFI significatively inhibited the ability of the two types of endothelial cell to invade the Matrigel™ and reach the abluminal compartment of the transwell. This result confirms that the peptide from the present invention conserves the vasoinhibin property with respect to inhibition of migration of endothelial cells.

### Example 3. Induction of the Expression of Vasoinhibin Target Genes

Vasoinhibin induces the expression of a variety of genes via the activation of NF-KB to promote angiostatic and inflammatory effects. In particular, interleukin-1 alpha (IL-1α) and the intercellular adhesion molecule 1 (ICAM1) are vasoinhibin gene targets in the bovine endothelial cells. To evaluate the ability of the oligopeptide THGRGFI to induce the expression of IL-1α and ICAM1, BUVEC E6E7 cells were seeded on 12 well plates with F12K—10% FBS, grown to 80% confluency, and starved with low serum (0.1% FBS) culture medium for 24 h. Then, the cells were treated with 100 nM vasoinhibin or the oligopeptide THGRGFI. After 4 hours the RNA was extracted from the cells using Trizol (Invitrogen, Carlsbad, CA) and retrotranscribed using the High Capacity cDNA Reverse Transcription Kit (Applied Biosystems, Foster City, CA). The RT-PCR products were quantified in a Maxima SYBRgreen qPCR (Thermo Fisher Scientific) 10 μl final volume reaction mixture containing the template and 0.25 μM of each primer. The PCR amplification was done in the CFX96 Real Time PCR (BioRad), comprising a 10-minute denaturalization at 95° C., followed by 35 amplification cycles (95° C. for 10 sec, 58° C. for 30 sec, and 72° C. for 30 sec). The primers used were IL-1α forward (5′-TCAAGGAGAATGTGGTGATG-3′=SEQ ID NO 7) and IL-1α reverse (5′-CTGGAAGCTGTAATGTGCTG-3′=SEQ ID NO 8); and ICAM1 forward (5′-CGTTAAGCTACACCCACCTT-3′=SEQ ID NO 9) and ICAM1 reverse (5′-AGGTAAGGGTCTCCAT CACA-3′=SEQ ID NO 10). The PCR data was analyzed by the 2^{-ΔΔCT} method and the threshold of the cycles (CT) was normalized by the constitutive housekeeping gene cyclophilin A (PPIA). The primers for PPIA amplification were PPIA forward (5′-GGTTCCCAGTTTTTCATTTG-3′=SEQ ID NO 11) and PPIA reverse (5′-ATGGTGATCTTCTTGCTGGT-3′=SEQ ID NO 12).

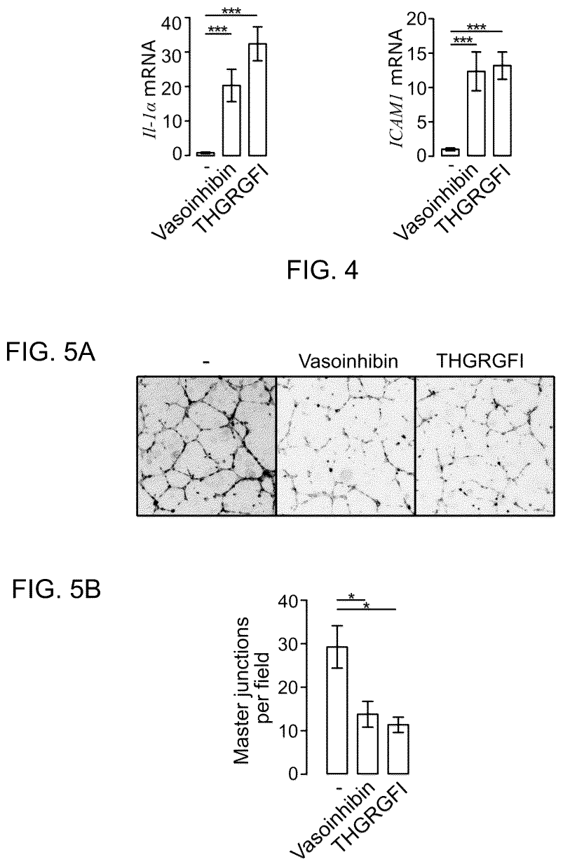

FIG. 4 shows the fold-change of expression of messenger RNA (mRNA) of vasoinhibin target genes interleukin-1α (IL-1α) and intracellular adhesion molecule 1 (ICAM1) in endothelial cells from the bovine umbilical vein (BUVEC E6E7) in response to 100 nM of a 123 amino acid vasoinhibin or the oligopeptide THGRGFI (***P<0.001). Vasoinhibin increased the mRNA levels of IL-1α and ICAM-1˜20-fold and ˜12 fold, respectively, versus the non-treated control whereas the oligopeptide THGRGFI increased the mRNA levels of IL-1α and ICAM ˜30-fold and ˜13-fold, respectively. These findings show that the oligopeptide of this invention conserves the vasoinhibin ability with respect to the induction of the expression of IL-1α and ICAM1.

### Example 4. Inhibition of the Formation of Capillary Structures

The formation of capillary structures is a late step in the angiogenesis process involving the migration, interaction, and organization of endothelial cells into tube-like capillary structures. Vasoinhibin disrupts this morphogenic process.

For investigating whether the oligopeptide THGRGFI shares this property, a primary culture of human endothelial cells from the umbilical-vein (HUVEC) was maintained in F12K media supplemented with 20% FBS, 100 μg ml⁻¹ of heparin, and 25 μg ml⁻¹ endothelial cell growth supplement (ECGS). Cells from passages 2 to 4 were rinsed with tempered PBS1x and were detached from the plate with 0.25% trypsin-EDTA for ˜3 minutes and centrifuged to remove trypsin. The cells were counted using an hemocytometer and seeded at a density of 29,000 cells cm⁻² in 300 μl of F12K medium supplemented with 20% FBS and with heparin on a 24-well plate, over a layer of ˜9.7 μg μl⁻¹ Matrigel™ which was previously polymerized for 1 hour at 37° C. Cells were then treated with 100 nM vasoinhibin or the oligopeptide THGRGFI, and after 6 hours, micrographs were obtained in an inverted microscope. The micrographs are shown in FIG. 5A. The images were analyzed to quantify main joining areas per field using the software “Angiogenesis Analyzer” [Gilles Carpentier. ImageJ contribution: Angiogenesis Analyzer. ImageJ News, 5 Oct. 2012.] and the ImageJ software.

Results are shown in FIG. 5B, *P<0.001. The capillary structures form spontaneously in response to angiogenic factors in the culture medium and the interaction of the cells with Matrigel™ components. Vasoinhibin and the oligopeptide THGRGFI disrupted the capillary structures, confirming that the oligopeptide of the invention conserves this vasoinhibin property.

### Example 5. Inhibition of Vasopermeability In Vitro

It is known that vasoinhibin inhibits vasopermeability by direct action on endothelial cells through the inactivation of the endothelial nitric oxide synthase (eNOS) in response to different vasoactive substances. This effect was demonstrated in vitro via confluent monolayers of endothelial cells derived from the bovine aorta and umbilical vein, the rat retinal capillaries, and the murine brain and retinal endothelial cells. Permeability was tested by measuring the transit of a big protein (radish peroxidase) through the endothelial cell monolayer or by changes in the transendothelial resistance (TEER) in the presence of different vasopermeability inducing factors. Vasoinhibin blocks the activation of eNOS through signaling pathways that involve the stimulation of the protein 2A phosphatase, which dephosphorylates/inactivates eNOS and by the blockage of the PLC and IP3 system and transient receptor potential (TRP) channels that reduce the intracellular calcium levels required for the calcium-calmodulin binding activation of eNOS.

For evaluating if the oligopeptide THGRGFI conserves the inhibitory properties of vasoinhibin on vascular permeability, the transit of Evans blue linked albumin across an endothelial cell (BUVEC E6E7) monolayer was evaluated as follows:

BUVEC E6E7 cells were seeded over a transwell filter (pore of 0.4 μm) at a density of 10,000 cells cm⁻². After 3 days, the monolayers were starved with low serum (0.1 FBS) for 48 hours. Subsequently, 100 nM of the vasoinhibin or the oligopeptide of the present invention were added to the upper compartment (luminal part) of the transwell support, incubated for 1 hour, and followed by the addition of 50 ng ml⁻¹ of VEGF. Control (Ctl) was without VEGF, vasoinhibin and the oligopeptide. After 10 minutes, the upper (luminal) culture media was substituted by 300 μl of PBS containing Evans blue-linked albumin and the medium of the inferior abluminal compartment was changed to 700 μl of PBS. Samples of 50 μl of the abluminal compartment were collected and replaced by fresh PBS at 10, 20, 30 and 60 minutes. The absorbance (620 nm) was measured at all time points using a plate reader iMARK (BioRad). Absorbance values confirmed the transit of Evans blue labeled albumin across the endothelial monolayer.

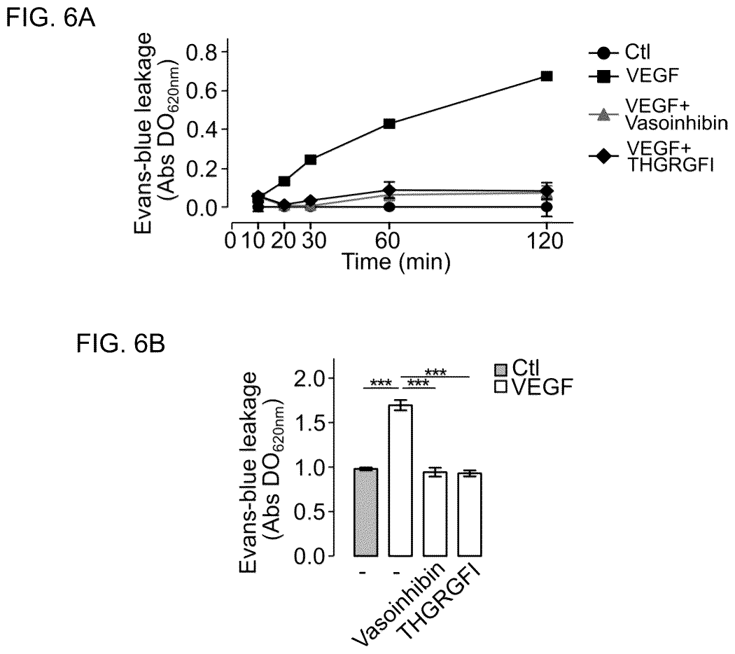

FIG. 6A shows the inhibition of vascular permeability by the 7 amino acid oligopeptide of the present invention and the 123 amino acids vasoinhibin throughout time (FIG. 6A) and at 120 minutes (FIG. 6B) (***P<0.001). As expected, VEGF stimulated the permeability of the endothelial monolayer and this effect increased over the time (FIG. 6A). Equal concentrations of vasoinhibin and of the oligopeptide THGRGFI inhibited the VEGF-induced increase in endothelial permeability, indicating that the peptide of the present invention conserves the ability of vasoinhibin to inhibit vasopermeability.

Another conventional protocol to evaluate vasopermeability is the measurement of the transendothelial electrical resistance (TEER), which uses a device that applies an electric current through electrodes at both sides of the endothelial cell monolayer. A reduction of the electrical resistance reveals a loss of barrier function and, consequently, an increase in permeability. The effect of the oligopeptide THGRGFI on endothelial cell permeability was evaluated by measuring TEER. BUVEC E6E7 were seeded in the TEER device at a density of 10,000 cells cm⁻². After three days, the monolayers were starved in low serum media (0.1% FBS) for 48 hours and after this time, 100 nM of vasoinhibin or of the oligopeptide were added to the upper compartment (luminal surface of the monolayer) for 1 hour. At time 0, TEER was recorded and 50 ng ml⁻¹ of VEGF were added to the luminal side. The TEER was measured at 10, 20, 30, 60, 90 and 120 minutes. Determinations were performed with the Epithelial Volt/Ohm (TEER) EVOM2 equipment (World Precision Instruments, FL, USA) with a 4 mm ‘chopstick’ electrode. Values were normalized relative to a device with no cells and a monolayer without treatment.

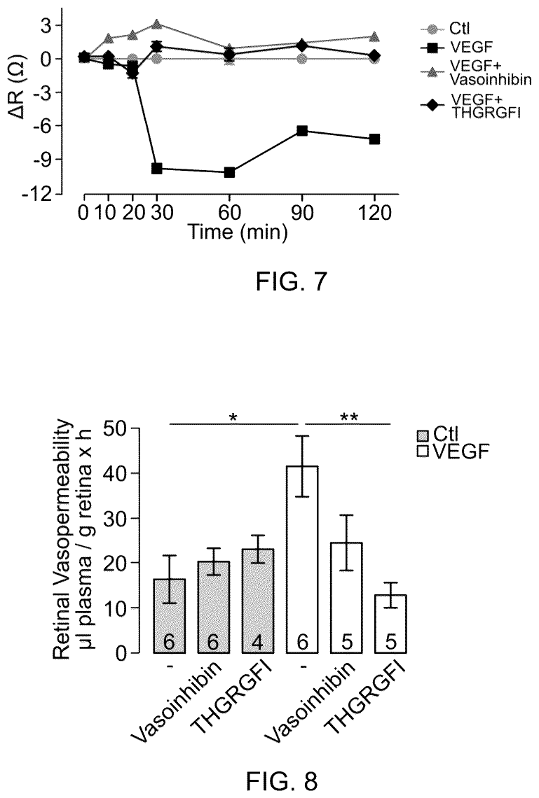

FIG. 7 shows the results. Vascular permeability was inhibited by the 7 amino acids oligopeptide and the vasoinhibin of 123 amino acids throughout time. The permeability was determined in absence (control, Ctl) or presence of VEGF alone or in combination with the oligopeptide of the invention or with vasoinhibin. The results show the expected reduction of transendothelial resistance by VEGF and that, both, the oligopeptide THGRGFI and vasoinhibin block the VEGF effect in a similar manner.

### Example 6. Inhibition of Vasopermeability In Vivo

Diabetic retinopathy and diabetic macular edema are the main causes of vision loss in diabetes and their early signs are an exacerbated vasopermeability in the retina. VEGF is the major factor responsible for these vascular changes, hence, the current therapy is based on the intravitreal injection of anti-VEGF antibodies able to neutralize VEGF. Vasoinhibin inhibits the increase in retinal vasopermeability in response to VEGF administered intravitreally, as well as the excessive vasopermeability due to diabetes in experimental models. The recombinant vasoinhibin protein and the vasoinhibin gene transduction with recombinant viral vectors have been used in these studies.

For evaluating if the oligopeptide THGRGFI conserves the inhibitory properties of vasoinhibin on VEGF-induced vasopermeability in vivo, the effect of the intravitreal injection of VEGF alone or in combination with the oligopeptide or vasoinhibin in rats was determined.

Wistar rats were intravitreally injected with saline (control) or with 300 ng of VEGF in each case alone or in combination with 20 μM of the oligopeptide of the invention or with 20 μM of vasoinhibin. After 24 hours, the extravasation of albumin into the retina was evaluated using the Evans blue method. Briefly, 45 mg kg⁻¹ of total weight of the Evans blue dye was injected intravenously to anesthetized rats and was allowed to circulate for 2 hours. Then, the animals were perfused with ˜80 ml of PBS at a flow of ˜40 ml min⁻¹. The retinas were dissected, dried and incubated with 200 μl of formamide (Mallinckrodt Baker, Phillipsburg, NJ) at 72° C. and after 18 hours the labeled albumin was determined in the retinal extracts.

Results are shown in FIG. 8 (*P<0.05, **P<0.02). The results showed that the intravitreal administration of the oligopeptide of the present invention blocks the VEGF increase in retinal vasopermeability in a similar way than vasoinhibin. From the fact that the treatment with VEGF-blocking antibodies is effective as a conventional therapy for diabetic retinopathy, the diabetic macular edema, and other vasoproliferative retinopathies (premature retinopathy and age-related macular degeneration), it is evident that the oligopeptide of the present invention has potential therapeutic value in these diseases.

### Example 7. Structural Characterization of the Oligopeptide THGRGFI

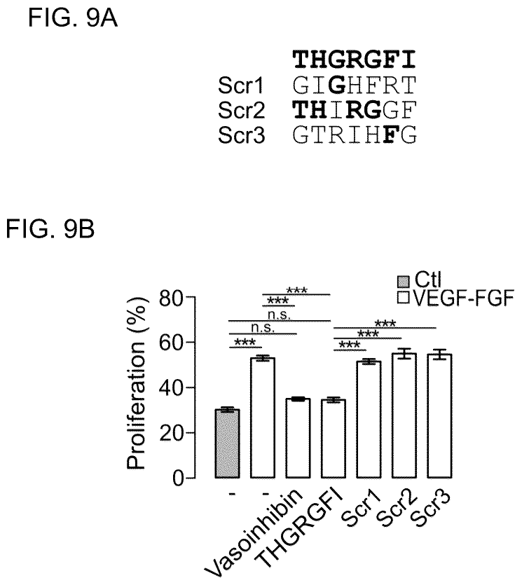

To determine if the antiangiogenic activity of the oligopeptide of the invention is specific to its sequence and not due to the amino acidic composition, 3 scrambled sequences were generated with the amino acids contained in the oligopeptide and tested for their effect on the proliferation of HUVEC cells at a concentration of 100 nM. The 3 sequences are GIGHFRT (SEQ ID NO 13), THIRGGF (SEQ ID NO 14) and GTRIHFG (SEQ ID NO 15). They are illustrated in FIG. 9A and designated as Scr1, Scr2 an Scr3 in FIGS. 9A and 9B. The amino acids that are not changed in their position are indicated in bold.

HUVEC were seeded on a 96 well plate at a density of ˜11,000 cells cm⁻² and maintained in F12K supplemented with 20% of FBS, 100 μg ml⁻¹ heparin and 25 μg ml⁻¹ endothelial cell growth supplement (ECGS). After 24 hours, the cells were synchronized in GO with starving conditions (FBS 0.5%) for 16 hours, and after that FBS and the heparin were replenished. Cells were then treated with THGRGFI, the different oligopeptides and 123 amino acids vasoinhibin, each in a concentration of 100 nM, for 24 hours in presence or absence of 10 μM of thymidine analog 5-Ethynyl-2′-deoxyuridine (EdU) and a combination of 25 ng ml⁻¹ VEGF and 20 ng ml⁻¹ bFGF. Finally, the cells were fixed with 4% paraformaldehyde, permeabilized with 0.5% of Triton X-100 in TBS1x and stained for the newly synthetized DNA with the ‘click’ method that detects the EdU incorporation. Total DNA was stained with Hoechst 33342 and the percentage of ‘click’-stained nuclei relative to total nuclei (Hoechst 33342-stained) indicated cell proliferation.

FIG. 9B shows the effects of the same concentration (100 nM) of 3 scrambled oligopeptides, of the 7 residues peptide THGRGFI of the invention and the 123 amino acids vasoinhibin on the proliferation of HUVEC stimulated with VEGF (25 ng ml⁻¹) and bFGF (20 ng ml⁻¹), ***P<0.001.

Through the scrambled oligopeptides shared the same aminoacidic composition with the oligopeptide of the present invention, none of the scrambled oligopeptides had an inhibitory effect on the proliferation of the endothelial cells. This indicates that the amino acid sequence is important for the activity of the oligopeptide of the invention.

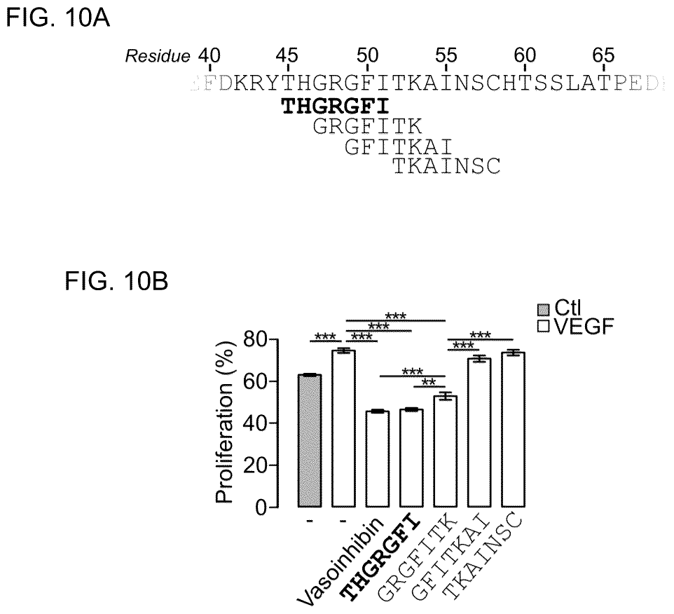

For understanding if the sequence of the peptide of the present invention is determinant for the vasoinhibin effect on the proliferation of endothelial cells or if this effect is still present in a neighboring sequence in the sequence of vasoinhibin, the inhibitory effects of 7 amino acid-oligopeptides shifted in the vasoinhibin sequence by 2 or 3 residues was evaluated. FIG. 10A shows the location the 7-residues oligopeptide THGRGFI of the present invention in the linear sequence of vasoinhibin. The amino acids and numbers are indicated in the sequence. The three oligopeptides SEQ ID NO 16, SEQ ID NO 17 and SEQ ID NO 18 of 7 amino acids having an overlapping sequence with the peptide of the present invention are also shown in FIG. 10A.

For the assay, BUVEC E6E7 cells were seeded at a density of ˜14′000 cells cm⁻² in a 96 well plate with F12K with 10% (v/v) FBS. After 24 hours, cells were starved with 0.1% of FBS for GO synchronization for ˜16 hours. Next, the cells were treated with the different oligopeptides in presence of 10 μM of the thymidine analog EdU and 50 ng ml⁻¹ of VEGF. Finally, the cells were fixed, permeabilized, and stained for the newly synthetized DNA by the EdU incorporation through the ‘click’ assay. The total DNA was stained with Hoechst 33342 and the percentage of ‘click’-stained nuclei over the total nuclei was a measure of proliferation.

FIG. 10B shows the effects of the same concentration (100 nM) of the shifted 7-residues oligopeptides, of the peptide of the present invention and of 123-residues vasoinhibin in the presence of VEGF (50 ng ml-1) in relation to VEGF (50 ng ml-1) alone and in the absence of VEGF and the oligopeptides (control, Ctl) on the proliferation of immortalized endothelial cells from the bovine umbilical vein (BUVEC E6E7), ***P<0.001.

The oligopeptide GRGFITK (SEQ ID NO 16), which is shifted by 2 residues in relation to THGRGFI significatively inhibited the VEGF-induced proliferation of endothelial cells. However, the inhibition was significatively lower than that by vasoinhibin and that by THGRGFI. The other 2 oligopeptides (GFITKAI (SEQ ID NO 17) and TKAINSC (SEQ ID NO 18)) did not shown any activity.

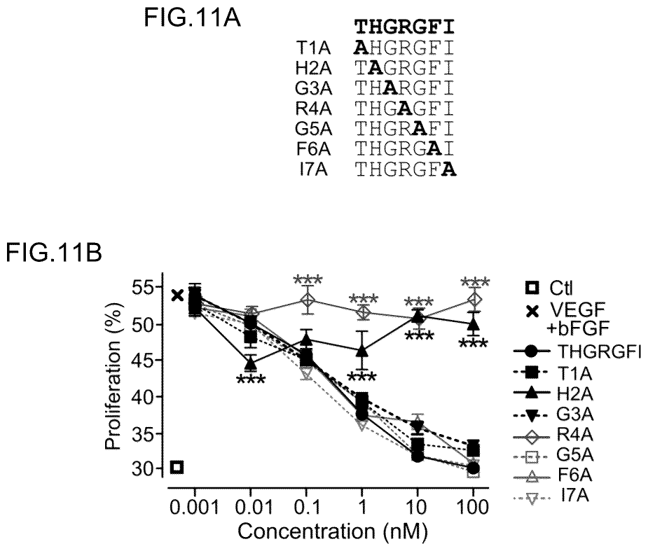

For evaluating the contribution of each of the amino acids to the inhibitory potency of the oligopeptide THGRGFI on the proliferation of HUVEC, an alanine scanning was performed with the 7 amino acids peptide and the dose-response effects of the various alanine substitutions on the proliferation of HUVEC were evaluated. FIG. 11A shows 7 sequences of synthetic oligopeptides of 7 amino acids in which oligopeptides a consecutive substitution of each amino acid by alanine was performed. The 7 sequences SEQ ID NO 19 to SEQ ID NO 25 are shown below the sequence of THGRGFI. The substituted amino acids are indicated in bold.

Approximately 11,000 HUVEC cells cm⁻² were seeded on a 96 well plate in F12K media with 20% FBS, 100 μg ml⁻¹ heparin and 25 μg ml⁻¹ ECGS. After 24 hours, cells were starved with 0.5% of FBS for ˜16 hours, and after that time the FBS and heparin were added again and cells treated with different doses of the alanine substituted oligopeptides for 24 hours, in the presence of EdU and a combination of 25 ng ml⁻¹ VEGF and 20 ng ml⁻¹ bFGF. Finally, the cells were fixed, permeabilized and stained to quantify the synthesis of DNA as a sign of proliferation. FIG. 11B shows the biological potency of the different oligopeptides indicated in FIG. 11A on the proliferation of the HUVEC cells stimulated with the combination of VEGF and bFGF, ***P<0.001.

The dose-response effect of most of the oligopeptides with the alanine-scanning mutations was similar to the oligopeptide of the invention THGRGFI, except that of the oligopeptides that suffered a mutation in the histidine residue at position X2 (H2A) and in the arginine at position X4 (R4A). These oligopeptides did not inhibit endothelial proliferation, indicating that amino acids at positions X2 and X4 are important amino acids mediating the inhibitory activity of the oligopeptide of the invention.

Since the histidine in X2 (H2) and the arginine X4 (R4) seems to be important for the activity of the oligopeptide of the invention and since the scramble peptide Scr2 of FIGS. 9A and 9B did not have any effect in spite of having the histidine in X2 and the arginine in X4, it seems that the glycine in position X3 has also a role in the bioactivity. These results imply that the peptide THGRGFI can be miniaturized even more. Accordingly, 2 peptides of 4 and 3 amino acids, THGR (SEQ ID NO 4) and HGR (SEQ ID NO 7), were synthetized and their biological potencies on the proliferation of HUVECs were tested.

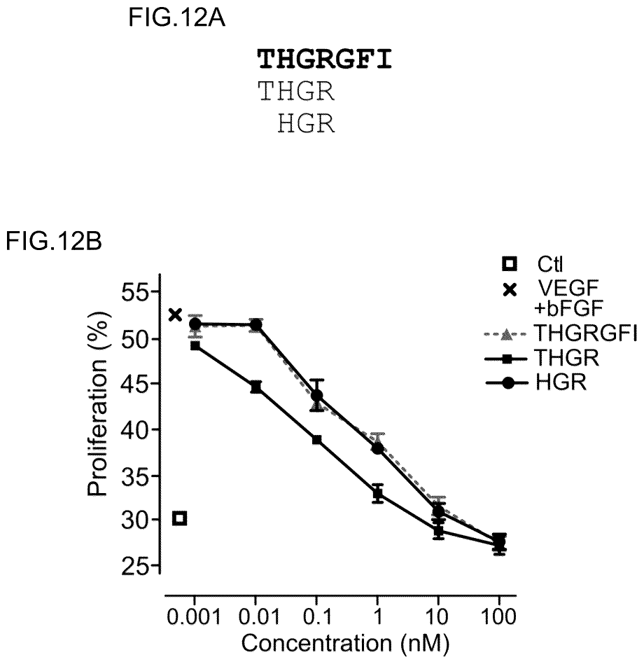

The FIG. 12A shows the sequences of oligopeptides of 7, 4 and 3 amino acids of the present invention. FIG. 12B compares the biological potencies of the oligopeptides indicated in FIG. 12A on the proliferation of human umbilical-vein endothelial cells stimulated with a combination of VEGF (25 ng ml⁻¹) and bFGF (20 ng ml⁻¹).

Both, the tetrapeptide THGR as well as the tripeptide HGR, behaved as vasoinhibin in the dose-response curve very similar to the oligopeptide THGRGFI.

### USES OF THE INVENTION (INDUSTRIAL APPLICABILITY)

The oligopeptide of the present invention and its respective pharmaceutical composition that inhibit angiogenesis and vascular function can be used to prevent or treat any condition or disease associated to excessive angiogenesis and vasopermeability. These diseases include tumor growth, rheumatoid arthritis, arteriosclerotic plaques formation, cornea neovascularization, proliferative retinopathies as diabetic retinopathy and macular degeneration, deficient wound repair, glaucoma, psoriasis, chronic varicose ulcers, reproductive disorders like follicular cysts, among others. Likewise, the oligopeptide can be used as contraceptive agent.

Due to its antiangiogenic properties, the oligopeptide of the present invention can be used to regulate blood vessel-dependent pathologic growth of organs and tissues. For example, the oligopeptide of the present invention can be used to inhibit the vascularization of tumors in order to reduce their size and facilitate their regression. Also, the oligopeptide of the present invention can be used to prevent and avoid the metastasis.

The oligopeptide of the present invention can be used as template for peptidomimetic protocols to generate peptidic or non-peptidic analogs or agonists of the actions of the oligopeptide. Modifications that can be done involve mutations or amino acid substitution with L-amino acids or with non-peptidic molecules. Furthermore, the oligopeptide can be further modified by miniaturization techniques or by the generation of constraint peptide like circular or retro-inverse oligopeptide.

The oligopeptide of the invention can be used as a template in peptidomimetic techniques to generate peptidic or non-peptidic antagonists for the blockage of the effects of the endogenous vasoinhibin. Modifications that can be performed involve mutations and substitutions with homolog residues, with L-amino acids, or non-peptidic structures, miniaturization techniques or the generation of constraint peptides such as circular or retro-inverse peptides, etc. The antagonist based on the oligopeptide of this invention can be used in treatment of a disease that involves an elevated level of endogenous vasoinhibin such as peripartum cardiomyopathy, preeclampsia, a condition related to abnormal blood pressure, a depressive disorder, an anxiety disorder, or delayed fetus growth.

The oligopeptide of the present invention provides information for the generation of methods that allow the quantification of the endogenous levels of vasoinhibin in blood, other body fluids, or tissues. For example, a radioimmuno assay or a sandwich type or Multiplex-type ELISA can be implemented. Any technique in the field to generate a diagnostic tool can be used. Today, the limiting issue for the development of this kind of assays is to generate antibodies that recognize the vasoinhibin but not the PRL. With the oligopeptide of the invention, rationalized antibodies that recognize the specific domain of vasoinhibin could be generated. Any antibody generated that recognizes the oligopeptide of the present invention can be used in a diagnostic method and is in the scope of the present invention. The diagnostic method for specific detection of vasoinhibin is of particular interest in the diagnosis of a reproductive disorder previously mentioned (preeclampsia, peripartum cardiomyopathy, delayed fetus growth), in a condition related to an abnormal blood pressure, in a depressive disorder, in an anxiety disorder or in an angiogenesis-dependent disease.

The oligopeptide of the present invention can be used for the treatment of the cardiovascular diseases, ischemic stroke and thrombosis. Vasoinhibin acts through binding to the plasminogen-activator inhibitor 1 (PAI-1) and antagonizes its effects, which have been related to thrombosis. Furthermore, the angiogenesis inhibition by different agents have been correlated to an increased risk of thromboembolism. The dual effects of the oligopeptide according to the invention, namely antiangiogenic effect and profibrinolytic effect, help to avoid secondary thrombo-generating effects.

Vasoinhibin has antimetastatic effects. The oligopeptide of the present invention can be used to block the invasion of cancer cells in the metastasis process. Furthermore, the oligopeptide can act on cancer cells to directly block their proliferation and migration. Therefore, the antitumor effect of the oligopeptide is dual as it can involve the blockage of tumor angiogenesis and the direct inhibition of tumor cell proliferation and migration.

The oligopeptide of the present invention can be used in combination with another protein such as an antibody or another antiangiogenic protein as a fusion protein. Moreover, it can be used as a ‘linker’ between two or more proteins, related or unrelated to angiogenesis. Likewise, the sequence or the elements of the oligopeptide of this invention can be converted to non-peptidic molecules through peptidomimetic strategies.

The oligopeptide of the present invention can be used to reduce the establishment of tumor metastasis.

The oligopeptide of this invention can be used to stimulate the fibrinolysis in thrombotic diseases and hemostasis alterations and cicatrix formation.

The oligopeptide of the present invention can also be used in veterinary, e.g., in the treatment of angiogenic-dependent diseases such as dog or cat cancer and other diseases of this kind in farm or domestic animals.

The features of the invention can be used individually or in any arbitrary combination. It is to be understood that the embodiments of the present invention are just illustrative and do not pretend to limit the scope of the present invention.

1. An oligopeptide that for inhibition of angiogenesis and vascular function which oligopeptide has a length of 3 to 7 amino acids and comprises or consists of
the sequence X2-X3-X4, wherein
X2 is a basic amino acid or an amide amino acid,
X3 is Ala or Gly and
X4 is a basic amino acid, charged at neutral pH
or
the sequence X1-X2-X3-X4, wherein
X1 is a polar non-charged amino acid and
X2, X3 and X4 are identical to X2, X3 and X4 in X2-X3-X4,
or
the sequence X1-X2-X3-X4-X5-X6-X7, wherein
X1, X2, X3 and X4 are identical to X1, X2, X3 and X4 in X1-X2-X3-X4,
X5 is Gly, Ser, or Ala,
X6 is a hydrophobic amino acid and
X7 is a hydrophobic amino acid, wherein
X1 is Thr, Ser, Asn, Glu, Gly, or Ala,
X2 is His, Am, Lys, Gln, or Asn,
X6 is Phe, Ala, Leu, Ile, Trp or Pro and
X7 is Phe, Ala, Leu, Ile, Trp or Pro,
wherein X4 is Lys for use in the treatment or prevention of a vasoproliferative retinopathy, diabetic retinopathy, rheumatoid arthritis, arteriosclerotic plaques formation, cornea neo-vascularization, macular degeneration, deficient wound repair, glaucoma, psoriasis, chronic varicose ulcers or follicular cysts,
or
wherein X4 is Arg for use in the treatment or prevention of cancer, a vasoproliferative retinopathy, diabetic retinopathy, rheumatoid arthritis, arteriosclerotic plaques formation, cornea neo-vascularization, macular degeneration, deficient wound repair, glaucoma, psoriasis, chronic varicose ulcers or follicular cysts.

2. The oligopeptide according to claim 1 for use in the treatment or prevention of cancer, a vasoproliferative retinopathy, diabetic retinopathy, rheumatoid arthritis, arteriosclerotic plaques formation, cornea neo-vascularization, macular degeneration, deficient wound repair, glaucoma, psoriasis, chronic varicose ulcers or follicular cysts, wherein
X1 is Thr,
X2 is His,
X3 is Gly,
X4 is Arg,
X5 is Gly,
X6 is Phe and
X7 is Ile.

3. An oligopeptide for inhibiting angiogenesis and vascular function which oligopeptide has a length of 3 to 7 amino acids and comprises or consists of
the sequence X1-X2-X3, wherein
X1 is an acid amino acid with negative charge at neutral pH,
X2 is a polar amino acid and
X3 is an amino acid with positive charge at neutral pH
or
the sequence X1-X2-X3-X4, wherein
X1, X2 and X3 are identical to X1, X2 and X3 in X1-X2-X3 and
X4 is a polar aromatic amino acid
or
the sequence X1-X2-X3-X4-X5-X6-X7, wherein
X1, X2, X3 and X4 are identical to X1, X2, X3 and X4 in X1-X2-X3-X4,
X5 is a polar amino acid,
X6 is a hydrophobic amino acid and
X7 is a hydrophobic amino acid,
wherein
X1 is Asp or Glu,
X2 is Gln, Asn, Ser or Thr,
X3 is Arg or Lys,
X4 is Tyr,
X5 is Gln, Asn, Ser or Thr,
X6 is Phe, Ala, Leu, Ile, Trp or Pro and
X7 is Phe, Ala, Leu, Ile, Trp or Pro.

4. The oligopeptide according to claim 3, wherein
X1 is Glu,
X2 is Gln,
X3 is Lys,
X5 is Ser,
X6 is Phe and
X7 is Leu.

5. An oligopeptide for inhibiting angiogenesis and vascular function which has a length of 7 amino acids and the sequence X1-X2-X3-X4-X5-X6-X7, wherein
X1 is the amino acid Thr, Asp or Glu,
X2 is the amino acid His or Gln,
X3 is the amino acid Gly or Lys,
X4 is the amino acid Arg if X3 is Gly or the amino acid Tyr if X3 is Lys,
X5 is the amino acid Gly or Ser,
X6 is the amino acid Phe and
X7 is the amino acid Ile or Leu.

6. The oligopeptide according to claim 1 for use in the treatment or prevention of cancer, a vasoproliferative retinopathy, diabetic retinopathy, rheumatoid arthritis, arteriosclerotic plaques formation, cornea neo-vascularization, macular degeneration, deficient wound repair, glaucoma, psoriasis, chronic varicose ulcers or follicular cysts which oligopeptide consists of or comprises any of sequences Thr His Gly Arg Gly Phe Ile (SEQ ID NO 1), Glu Gln Lys Tyr Ser Phe Leu (SEQ ID NO 2), Asp Gln Lys Tyr Ser Phe Leu (SEQ ID NO 3), Thr His Gly Arg (SEQ ID NO 4), Glu Gln Lys Tyr (SEQ ID NO 5), Asp Gln Lys Tyr (SEQ ID NO 6), His Gly Arg, Glu GlnLys, and Asp Gln Lys.

7. The oligopeptide according to claim 1 for use in the treatment or prevention of cancer, a vasoproliferative retinopathy, diabetic retinopathy, rheumatoid arthritis, arteriosclerotic plaques formation, cornea neo-vascularization, macular degeneration, deficient wound repair, glaucoma, psoriasis, chronic varicose ulcers or follicular cysts which oligopeptide consists of or comprises a sequence having at least 70%, in particular at least 80%, similarity to any of the sequences defined in claim 6, wherein similarity to an oligopeptide means percentage of amino acids of the total number of amino acids of the oligopeptide replaced by conservative replacement.

8. The oligopeptide according to claim 1, wherein the oligopeptide has a modification at one terminal end or modifications at both terminal ends of its sequence or a substitution of one, more or all of its amino acids having D-conformation by an amino acid or amino acids having L-conformation.

9. The oligopeptide according to claim 2 for use in the treatment or prevention of cancer, a vasoproliferative retinopathy, diabetic retinopathy, rheumatoid arthritis, arteriosclerotic plaques formation, cornea neo-vascularization, macular degeneration, deficient wound repair, glaucoma, psoriasis, chronic varicose ulcers or follicular cysts, wherein the oligopeptide has a modification at one terminal end or modifications at both terminal ends of its sequence or a substitution of one, more or all of its amino acids having D-conformation by an amino acid or amino acids having L-conformation.

10. The oligopeptide according to claim 8, wherein the modification(s) is/are acetylation of an N-terminal end and/or amidation of a C-terminal end of the oligopeptide or a covalent binding between an N-terminal amino acid and a C-terminal amino acid of the oligopeptide resulting in a cyclization of the oligopeptide.

11. (canceled)

12. A recombinant protein comprising a sequence of an oligopeptide according to claim 1, wherein X4 is Lys for use in the treatment or prevention of a vasoproliferative retinopathy, diabetic retinopathy, rheumatoid arthritis, arteriosclerotic plaques formation, cornea neo-vascularization, macular degeneration, deficient wound repair, glaucoma, psoriasis, chronic varicose ulcers or follicular cysts
or
wherein X4 is Arg for use in the treatment or prevention of cancer, a vasoproliferative retinopathy, diabetic retinopathy, rheumatoid arthritis, arteriosclerotic plaques formation, cornea neo-vascularization, macular degeneration, deficient wound repair, glaucoma, psoriasis, chronic varicose ulcers or follicular cysts.

13. A recombinant protein comprising a sequence of an oligopeptide according to claim 3 for use in the treatment or prevention of cancer, a vasoproliferative retinopathy, diabetic retinopathy, rheumatoid arthritis, arteriosclerotic plaques formation, cornea neo-vascularization, macular degeneration, deficient wound repair,
glaucoma, psoriasis, chronic varicose ulcers or follicular cysts.

14. A recombinant nucleic acid which nucleic acid consists of or comprises a sequence coding for an oligopeptide according to claim 3 or a sequence complementary to this sequence.

15. The recombinant nucleic acid according to claim 14, wherein the nucleic acid is contained in an expression vector.

16. A pharmaceutical composition comprising at least one oligopeptide according to claim 1 for the use according to the respective claim and/or at least on recombinant protein according to claim 12 the use according to the respective claim and/or at least one recombinant nucleic acid according to claim 14 for use in the treatment or prevention of cancer, a vasoproliferative retinopathy, diabetic retinopathy, rheumatoid arthritis, arteriosclerotic plaques formation, cornea neo-vascularization, macular degeneration, deficient wound repair, glaucoma, psoriasis, chronic varicose ulcers or follicular cysts.

17. The pharmaceutical composition according to claim 16 comprising a pharmaceutically acceptable carrier.

18. An antibody generated from the use of an oligopeptide according to claim 1 or the recombinant protein according to claim 12, wherein X4 is Lys, or according to claim 13.

19. The antibody of claim 18, wherein the oligopeptide comprises or consists of sequence His Gly Arg, Glu Gln Lys, or sequence Asp Gln Lys and the recombinant protein comprises one of sequences Glu Gln Lys and Asp Gln Lys.

20. The antibody of claim 18, wherein the antibodies are configured for use in a diagnostic method performed in vitro.

21. The antibody of to claim 20, wherein the diagnostic method is configured for diagnosis of preeclampsia, peripartum cardiomyopathy, delayed fetus growth, a condition related to an abnormal blood pressure, a depressive disorder, an anxiety disorder, or an angiogenesis-dependent disease, wherein the angiogenesis-dependent disease is cancer, a vasoproliferative retinopathy, diabetic retinopathy or rheumatoid arthritis.


SEQUENCE LISTING (25 sequences)

SEQ ID NO: 1 - protein, 7 aa, Homo sapiens
  Amino acids 45 to 51 of human prolactin
THGRGFI

SEQ ID NO: 2 - protein, 7 aa, Homo sapiens
  Amino acids 39 to 45 of human growth hormone
EQKYSFL

SEQ ID NO: 3 - protein, 7 aa, Homo sapiens
  Amino acids 39 to 45 of human placental lactogen
DQKYSFL

SEQ ID NO: 4 - protein, 4 aa, Homo sapiens
  Amino acids 45 to 48 of human prolactin
THGR

SEQ ID NO: 5 - protein, 4 aa, Homo sapiens
  Amino acids 39 to 42 of human growth hormone
EQKY

SEQ ID NO: 6 - protein, 4 aa, Homo sapiens
  Amino acids 39 to 42 of human placental lactogen
DQKY

SEQ ID NO: 7 - DNA, 20 bp, Artificial Sequence
  PCR primer
tcaaggagaatgtggtgatg

SEQ ID NO: 8 - DNA, 20 bp, Artificial Sequence
  PCR primer
ctggaagctgtaatgtgctg

SEQ ID NO: 9 - DNA, 20 bp, Artificial Sequence
  PCR primer
cgttaagctacacccacctt

SEQ ID NO: 10 - DNA, 20 bp, Unknown
  PCR primer
aggtaagggtctccatcaca

SEQ ID NO: 11 - DNA, 20 bp, Artificial Sequence
  PCR primer
ggttcccagtttttcatttg

SEQ ID NO: 12 - DNA, 20 bp, Artificial Sequence
  PCR primer
atggtgatcttcttgctggt

SEQ ID NO: 13 - protein, 7 aa, Artificial Sequence
  scrambled sequence of SEQ ID NO 1
GIGHFRT

SEQ ID NO: 14 - protein, 7 aa, Artificial Sequence
  scrambled sequence of SEQ ID NO 1
THIRGGF

SEQ ID NO: 15 - protein, 7 aa, Artificial Sequence
  scrambled sequence of SEQ ID NO 1
GTRIHFG

SEQ ID NO: 16 - protein, 7 aa, Homo sapiens
  Amino acids 47 to 53 of human prolactin
GRGFITK

SEQ ID NO: 17 - protein, 7 aa, Homo sapiens
  Amino acids 49 to 55 of human prolactin
GFITKAI

SEQ ID NO: 18 - protein, 7 aa, Homo sapiens
  Amino acids 52 to 58 of human prolactin
TKAINSC

SEQ ID NO: 19 - protein, 7 aa, Artificial Sequence
  Position 1 of SEQ ID NO 1 replaced by alanine
AHGRGFI

SEQ ID NO: 20 - protein, 7 aa, Artificial Sequence
  Position 2 of SEQ ID NO 1 replaced by alanine
TAGRGFI

SEQ ID NO: 21 - protein, 7 aa, Artificial Sequence
  Position 3 of SEQ ID NO 1 replaced by alanine
THARGFI

SEQ ID NO: 22 - protein, 7 aa, Artificial Sequence
  Position 4 of SEQ ID NO 1 replaced by alanine
THGAGFI

SEQ ID NO: 23 - protein, 7 aa, Artificial Sequence
  Position 5 of SEQ ID NO 1 replaced by alanine
THGRAFI

SEQ ID NO: 24 - protein, 7 aa, Artificial Sequence
  Position 6 of SEQ ID NO 1 replaced by alanine
THGRGAI

SEQ ID NO: 25 - protein, 7 aa, Artificial Sequence
  Position 7 of SEQ ID NO 1 replaced by alanine
THGRGFA